(19) United States
(12) Patent Application Publication
(10) Pub. No.: US 2014/0141419 A1
(43) Pub. Date: May 22, 2014

(54) SINGLE TUBE QUANTITATIVE POLYMERASE CHAIN REACTION (PCR)

(71) Applicants: Jay Lewington, Bisley Surrey (GB); Rohit Mistry, Watford (GB); J. Aquilez Sanchez, Framingham, MA (US)

(72) Inventors: Jay Lewington, Bisley Surrey (GB); Rohit Mistry, Watford (GB); J. Aquilez Sanchez, Framingham, MA (US)

(73) Assignees: SMITHS DETECTION - WATFORD LIMITED, Watford (GB); BRANDEIS UNIVERSITY, Waltham, MA (US)

(21) Appl. No.: 13/979,323

(22) PCT Filed: Jan. 13, 2012
(86) PCT No.: PCT/US12/21322

§ 371 Date: Feb. 7, 2014

Related U.S. Application Data
(60) Provisional application No. 61/432,899, filed on Jan. 14, 2011.

(51) Int. Cl. C12Q 1/68; G06F 19/22

(52) U.S. Cl. CPC C12Q 1/686; G06F 19/22
USPC 435/6.11; 702/19

(57) ABSTRACT

Provided herein are systems and methods for quantitatively monitoring target amplicons produced by polymerase chain reaction (PCR). In particular, quantitative monitoring of target amplicon(s) in a single-tube PCR reaction without separate calibration reactions are provided.

### CROSS-REFERENCE TO RELATED APPLICATION

The present invention claims priority to U.S. Provisional Patent Application Ser. No. 61/432,899 filed Jan. 14, 2011, which is herein incorporated by reference in its entirety.

### FIELD

Provided herein are systems and methods for quantitatively monitoring target amplicons produced by polymerase chain reaction (PCR). In particular, quantitative monitoring of target amplicon(s) in a single-tube PCR reaction without separate calibration reactions are provided.

### BACKGROUND

Quantification of target amplicons in PCR reaction has proven technically challenging because the absolute fluorescence in a reaction is not directly related to the concentration of the dye present in a reporting configuration. Some researchers have sought to solve this problem by performing separate PCR reactions containing pre-calibrated standards in-parallel with the PCR reaction of interest. While successful, the need for multiple calibration reactions has proven to be cumbersome and inefficient.

### SUMMARY

Provided herein are methods for quantification of a nucleic acid amplification reaction, comprising: (a) combining one or more target nucleic acids, amplification reagents, control nucleic acids, and one or more quantification probes in a reaction vessel; (b) obtaining pre-amplification temperature-dependent detection signatures of the quantification probes; (c) performing amplification reaction; (d) obtaining post-amplification temperature-dependent signatures of the quantification probes; (e) normalizing the post-amplification signatures to the pre-amplification fluorescence signatures; and (f) quantifying target amplification based on the normalized signatures. In some embodiments, the nucleic acid amplification reaction comprises PCR. In some embodiments, PCR comprises asymmetric PCR (e.g. LATE-PCR). In some embodiments, quantification probes comprise one or more detectable labels. In some embodiments, quantification probes comprise one or more fluorescent labels. In some embodiments, quantification probes comprise molecular beacons. In some embodiments, obtaining temperature-dependent detection signatures comprises detecting one or more quantification probes at multiple temperatures (e.g. a range of temperatures (e.g. 5° C. to 99° C. or ranges therein). In some embodiments, obtaining said temperature-dependent detection signatures comprises detecting one or more quantification probes at three temperatures. In some embodiments, control nucleic acids are non-amplifiable in the amplification reaction. In some embodiments, quantification probes are configured to hybridize to one or more sequences in the control nucleic acids. In some embodiments, the quantification probes are configured to hybridize to sequences in two control nucleic acids at a first temperature (e.g. low temperature). In some embodiments, the quantification probes are configured to hybridize to sequences in one control nucleic acid at a second temperature (e.g. high temperature) that is higher than the first temperature. In some embodiments, said quantification probes are configured not to hybridize to the control nucleic acids at a third temperature (e.g. background-detection temperature) that is higher than the second temperature. In some embodiments, the quantification probes are configured to hybridize with one or more sequences suspected to be present in the target nucleic acid or known to be present in the target nucleic acid. In some embodiments, target nucleic acids are amplifiable by the amplification reagents in an amplification reaction. In some embodiments, normalizing comprises subtracting pre-amplification signatures from the post-amplification fluorescence signatures to generate a normalized signature. In some embodiments, quantifying target amplification comprises comparing the normalized signature to the pre-amplification signature. In some embodiments, pre-amplification temperature-dependent detection signatures are obtained prior to the addition of the enzyme responsible for amplification to the reaction vessel.

Provided herein are kits comprising: amplification reagents, one or more quantification probes, and a set of control nucleic acids, wherein the control nucleic acids are non-amplifiable by the amplification reagents. In some embodiments, the control nucleic acids are blocked at the 3′ end to prevent control nucleic acids from functioning as primers. In some embodiments, the amplification reagents comprise amplification primers. In some embodiments, the control nucleic acids are non-complementary to the 3′ end of the amplification primers. In some embodiments the one or more quantification probes is configured to hybridize sequences in two control nucleic acids in the set of control nucleic acids. In some embodiments, each of the one or more quantification probes is configured to hybridize to either of two control nucleic acids at a first temperature (e.g. low temperature). In some embodiments, each of the one or more quantification probes is configured to hybridize to only one of the two control nucleic acids at a second temperature (e.g. high temperature). In some embodiments, each of the one or more quantification probes is configured to hybridize to neither of the two control nucleic acids at a third temperature (e.g. background-detection temperature). In some embodiments, the third temperature is higher than the second temperature. In some embodiments, the second temperature is higher than the first temperature.

Provided herein are systems for analyzing the result of an amplification reaction comprising a processor configured for: (a) accepting an input comprising: (i) a first data set, wherein the first data set comprises pre-amplification signatures or a amplification reaction mixture; and (ii) a second data set, wherein the second data set comprises post-amplification signatures or a amplification reaction mixture; (b) calculating a normalized data set from the first data set and the second data set; (c) generating results by comparing the second data set to the normalized data set; and (d) reporting the results.

Additional embodiments are described herein. It should be understood that the descriptions of embodiments provided herein are illustrative embodiments and that one of skill in the art will appreciate and understand variations of these embodiments as being included within the scope of the inventions provided herein.

### DESCRIPTION OF THE FIGURES

FIG. 1 shows a schematic demonstrating LATE PCR assays for SNP genotyping.

FIG. 2 shows a schematic demonstrating genotyping of a SNP with LATE PCR

FIG. 3 shows a schematic demonstrating grouping of endpoint results.

FIG. 4 shows a schematic demonstrating generation of fluorescence rations from internal controls.

FIG. 5 shows a plot of pre-amplification and post-amplification fluorescence ratios.

FIG. 6 shows a plot demonstrating selection of temperatures for use in endpoint genotyping assay.

FIG. 7 shows a plot of pre-amplification first derivative fluorescence spectra.

FIG. 8 shows a plot of normalized pre-amplification fluorescence spectra.

FIG. 9 shows plots of post-amplification first derivative fluorescence spectra (top), and normalized post-amplification fluorescence spectra (bottom).

FIG. 10 shows a plot of the difference between pre-amplification fluorescence ratios and post-amplification fluorescence ratios.

To facilitate an understanding of specific embodiments described herein, a number of terms and phrases are defined below. It should be understood that embodiments are provided herein that are not limited to the embodiments described in these particular definitions.

As used herein, the term “molecular beacon probe” refers to a single-stranded oligonucleotide, typically 25 to 35 bases-long, in which the bases on the 3′ and 5′ ends are complementary forming a “stem,” typically for 5 to 8 base pairs. In certain embodiments, the molecular beacons employed have stems that are exactly 2 or 3 base pairs in length. A molecular beacon probe forms a hairpin structure at temperatures at and below those used to anneal the primers to the template (typically below about 60° C.). The double-helical stem of the hairpin brings a fluorophore (or other label) attached to the 5′ end of the probe very close to a quencher attached to the 3′ end of the probe. The probe does not fluoresce (or otherwise provide a signal) in this conformation. If a probe is heated above the temperature needed to melt the double stranded stem apart, or the probe is allowed to hybridize to a target oligonucleotide that is complementary to the sequence within the single-strand loop of the probe, the fluorophore and the quencher are separated, and the fluorophore fluoresces in the resulting conformation. Therefore, in a series of PCR cycles the strength of the fluorescent signal increases in proportion to the amount of the beacon hybridized to the amplicon, when the signal is read at the annealing temperature. Molecular beacons with different loop sequences can be conjugated to different fluorophores in order to monitor increases in amplicons that differ by as little as one base (Tyagi, S, and Kramer, F. R. (1996), Nat. Biotech. 14:303 308; Tyagi, S. et al., (1998), Nat. Biotech. 16: 49 53; Kostrikis, L. G. et al., (1998), Science 279: 1228 1229; all of which are herein incorporated by reference).

As used herein, the phrase “probe hybridization sequence” is used is reference to a particular target sequence and a particular probe, and it is the sequence in the target sequence that hybridizes to the particular probe. The probe may be fully or partially complementary to the target sequence over the length of the probe hybridization sequence. In some embodiments, the probe hybridization sequence is labeled to enable its detection (e.g., with a fluorophore at one end and quencher at the other end).

As used herein, the term “amplicon” refers to a nucleic acid generated using one or more primers, such as those described herein. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. In certain embodiments, the type of amplification is asymmetric PCR (e.g., LATE-PCR) which is described in, for example, U.S. Pat. No. 7,198,897, Sanchez et al., PNAS, 2004, 101(7):1933-1938, and Pierce et al., PNAS, 2005, 102(24):8609-8614, all of which are herein incorporated by reference in their entireties. In particular embodiments, LATE-PCR is employed using multiple end-point temperature detection (see, e.g., U.S. Pat. Pub. 2006/0177841 and Sanchez et al., BMC Biotechnology, 2006, 6:44, pages 1-14, both of which are herein incorporated by reference).

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the melting temperature (T_{M}) of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

In some embodiments, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, etc.) from one or more bioagents. Samples can include, for example, blood, saliva, urine, feces, anorectal swabs, vaginal swabs, cervical swabs, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxygenin; luminogenic, phosphorescent or fluorogenic moieties; and fluorescent dyes alone or in combination with moieties that can suppress (“quench”) or shift emission spectra by fluorescence resonance energy transfer (FRET). FRET is a distance-dependent interaction between the electronic excited states of two molecules (e.g., two dye molecules, or a dye molecule and a non-fluorescing quencher molecule) in which excitation is transferred from a donor molecule to an acceptor molecule without emission of a photon. (Stryer et al., 1978, Ann. Rev. Biochem., 47:819; Selvin, 1995, Methods Enzymol., 246:300, each incorporated herein by reference). As used herein, the term “donor” refers to a fluorophore that absorbs at a first wavelength and emits at a second, longer wavelength. The term “acceptor” refers to a moiety such as a fluorophore, chromophore, or quencher that has an absorption spectrum that overlaps the donor's emission spectrum, and that is able to absorb some or most of the emitted energy from the donor when it is near the donor group (typically between 1-100 nm). If the acceptor is a fluorophore, it generally then re-emits at a third, still longer wavelength; if it is a chromophore or quencher, it then releases the energy absorbed from the donor without emitting a photon. In some embodiments, changes in detectable emission from a donor dye (e.g. when an acceptor moiety is near or distant) are detected. In some embodiments, changes in detectable emission from an acceptor dye are detected. In some embodiments, the emission spectrum of the acceptor dye is distinct from the emission spectrum of the donor dye such that emissions from the dyes can be differentiated (e.g., spectrally resolved) from each other.

Labels may provide signals detectable by fluorescence (e.g., simple fluorescence, FRET, time-resolved fluorescence, fluorescence polarization, etc.), radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral.

“T_{M},” or “melting temperature,” of an oligonucleotide describes the temperature (in degrees Celsius) at which 50% of the molecules in a population of a single-stranded oligonucleotide are hybridized to their complementary sequence and 50% of the molecules in the population are not-hybridized to said complementary sequence. The T_{M} of a primer or probe can be determined empirically by means of a melting curve. In some cases it can also be calculated. For the design of symmetric and asymmetric PCR primer pairs, balanced T_{M}'s are generally calculated by one of the three methods discussed earlier, that is, the “% GC”, or the “2(A+T) plus 4 (G+C)”, or “Nearest Neighbor” formula at some chosen set of conditions of monovalent salt concentration and primer concentration. In the case of Nearest Neighbor calculations the T_{M}'s of both primers will depend on the concentrations chosen for use in calculation or measurement, the difference between the T_{M}'s of the two primers will not change substantially as long as the primer concentrations are equimolar, as they normally are with respect to PCR primer measurements and calculations. T_{M[}1] describes the calculated T_{M} of a PCR primer at particular standard conditions of 1 micromolar (1 uM=10⁻⁶M) primer concentration, and 0.07 molar monovalent cations. In this application, unless otherwise stated, T_{M[}1] is calculated using Nearest Neighbor formula, T_{M}=ΔH/(ΔS+R ln(C/2))−273.15+12 log [M]. This formula is based on the published formula (Le Novere, N. (2001), “MELTING, Computing the Melting Temperature of Nucleic Acid Duplex,” Bioinformatics 17: 1226 7). ΔH is the enthalpy and ΔS is the entropy (both ΔH and ΔS calculations are based on Allawi and SantaLucia, 1997), C is the concentration of the oligonucleotide (10⁻⁶M), R is the universal gas constant, and [M] is the molar concentration of monovalent cations (0.07). According to this formula the nucleotide base composition of the oligonucleotide (contained in the terms ΔH and ΔS), the salt concentration, and the concentration of the oligonucleotide (contained in the term C) influence the T_{M}. In general for oligonucleotides of the same length, the T_{M} increases as the percentage of guanine and cytosine bases of the oligonucleotide increases, but the T_{M} decreases as the concentration of the oligonucleotide decreases. In the case of a primer with nucleotides other than A, T, C and G or with covalent modification, T_{M[}1] is measured empirically by hybridization melting analysis as known in the art.

“T_{M[}0]” means the T_{M} of a PCR primer or probe at the start of a PCR amplification taking into account its starting concentration, length, and composition. Unless otherwise stated, T_{M[}0] is the calculated T_{M} of a PCR primer at the actual starting concentration of that primer in the reaction mixture, under assumed standard conditions of 0.07 M monovalent cations and the presence of a vast excess concentration of a target oligonucleotide having a sequence complementary to that of the primer. In instances where a target sequence is not fully complementary to a primer it is important to consider not only the T_{M[}0] of the primer against its complements but also the concentration-adjusted melting point of the imperfect hybrid formed between the primer and the target. In this application, T_{M[}0] for a primer is calculated using the Nearest Neighbor formula and conditions stated in the previous paragraph, but using the actual starting micromolar concentration of the primer. In the case of a primer with nucleotides other than A, T, C and G or with covalent modification, T_{M[}0] is measured empirically by hybridization melting analysis as known in the art.

As used herein superscript X refers to the Excess Primer, superscript L refers to the Limiting Primer, superscript A refers to the amplicon, and superscript P refers to the probe.

T_{M}^{A} means the melting temperature of an amplicon, either a double-stranded amplicon or a single-stranded amplicon hybridized to its complement. In this application, unless otherwise stated, the melting point of an amplicon, or T_{M}^{A}, refers to the T_{M} calculated by the following % GC formula: T_{M}^{A}=81.5+0.41(% G+% C)−500/L+16.6 log [M]/(1+0.7 [M]), where L is the length in nucleotides and [M] is the molar concentration of monovalent cations.

T_{M[}0]^{P} refers to the concentration-adjusted melting temperature of the probe to its target, or the portion of probe that actually is complementary to the target sequence (e.g., the loop sequence of a molecular beacon probe). In the case of most linear probes, T_{M[}0]^{P} is calculated using the Nearest Neighbor formula given above, as for T_{M[}0], or preferably is measured empirically. In the case of molecular beacons, a rough estimate of T_{M[}0]^{P} can be calculated using commercially available computer programs that utilize the % GC method, see Marras, S. A. et al. (1999) “Multiplex Detection of Single-Nucleotide Variations Using Molecular Beacons,” Genet. Anal. 14:151 156, or using the Nearest Neighbor formula, or preferably is measured empirically. In the case of probes having non-conventional bases and for double-stranded probes, T_{M[}0]^{P} is determined empirically.

C_{T} means threshold cycle and signifies the cycle of a real-time PCR amplification assay in which signal from a reporter indicative of amplicons generation first becomes detectable above background. Because empirically measured background levels can be slightly variable, it is standard practice to measure the C_{T} at the point in the reaction when the signal reaches 10 standard deviations above the background level averaged over the 5-10 preceding thermal cycles.

As used herein, the term “non-amplifiable” refers oligonucleotides which are not capable of being amplified by amplification reagents, typically due to lack of complementarity with amplification primers. In some embodiments, “non-amplifiable controls” refer to non-amplifiable oligonucleotides targets for the detection probe that are added to a PCR sample to generate reference fluorescent ratios/signals. In some embodiments, these oligonucleotides targets lack complementary to the primers used for PCR amplification and are therefore non-amplifiable.

### DETAILED DESCRIPTION

Provided herein are compositions, systems, kits, and methods for nucleic acid based diagnostic assays. In particular, probes and non-amplifiable control targets for amplification reactions, for example PCR (e.g. asymmetric PCR) and other amplification modalities are provided. In some embodiments, systems and methods for quantitatively monitoring target amplicons are provided. In particular, quantitative monitoring of target amplicon(s) in a single-tube PCR reaction without separate calibration reactions are provided. Provided herein are non-amplifiable control targets that are added to an amplification detection assay prior to amplification for use in generating reference probe signals or reference probe signal ratios. In some embodiments, methods for obtaining temperature-dependent detection signatures before and after amplification, and generating a normalized detection signature to quantitatively monitor target amplification are provided.

Embodiments herein find use in any application that identifies SNPs, other polymorphisms, or other sequences of interest. For example, provided herein are compositions and methods for use in screening and diagnostic assays that identify allelic imbalances due to chromosomal copy number variations (e.g. deletions, duplications), the presence of or identity of pathogenic nucleic acid in a sample, and the like. In some embodiments, controlled quantitative analysis of the allelic make-up of a SNP (e.g. homozygous, heterozygous) is provided. Additional uses are within the scope of one of skill in the art.

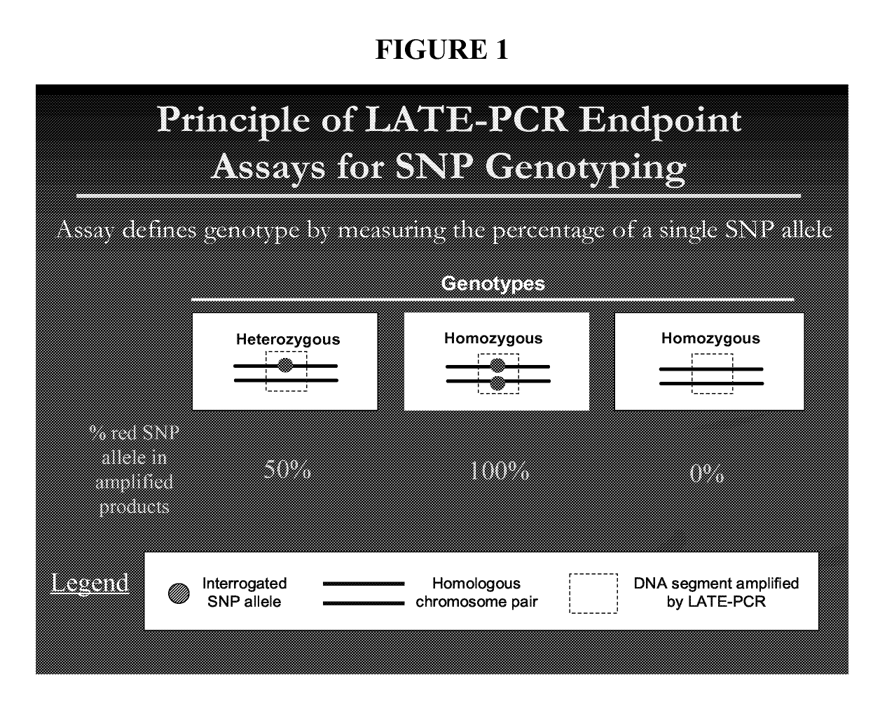

Benefits of the systems and methods are illustrated in the context of Three-Temperature LATE-PCT Genotyping. One form of LATE-PCR provides methods for endpoint genotyping of single nucleotide polymorphisms (SNP) termed Three-Temperature LATE-PCR Genotyping. The method identifies heterozygous and homozygous forms of a SNP allele by measuring the percentage of the interrogated SNP allele in the tested DNA sample. As shown in FIG. 1, the interrogated SNP allele (red spot in FIG. 1) is heterozygous if present in 50% of the tested DNA sample or it is homozygous if present in 100% of the tested DNA sample. The absence of the interrogated allele in the tested sample (0% detection) indicates that the sample is homozygous for the non-interrogated SNP allele, see FIG. 1.

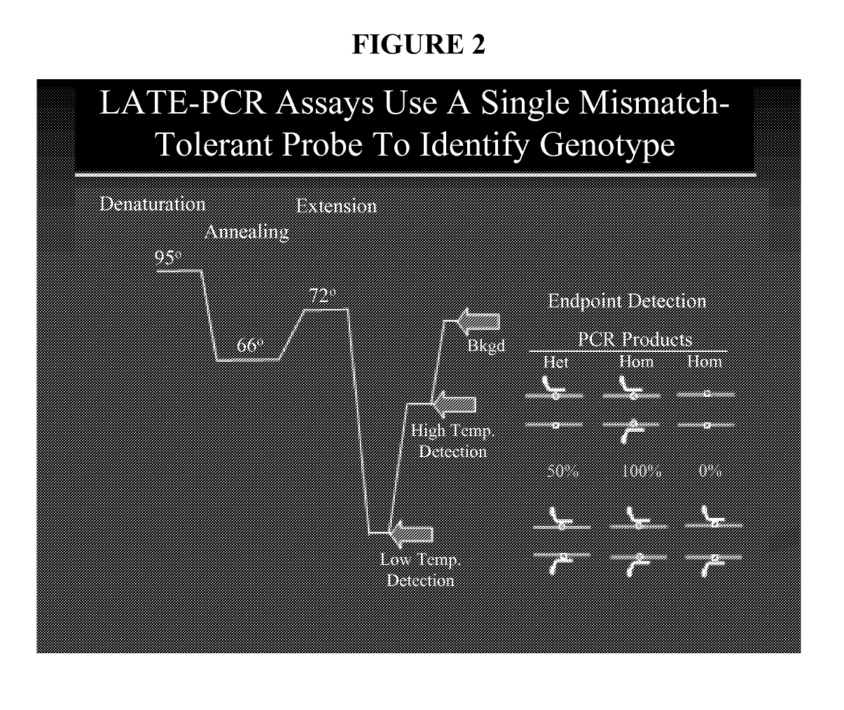

LATE-PCR endpoint genotyping assays typically use a single mismatch-tolerant fluorescent probe to measure the percentage of SNP allele percentages associated with each genotype. LATE-PCR generates large amounts of single-stranded DNA products that remain available for detection with the mismatch-tolerant probe over a large range of temperatures at the end of the amplification reaction. The single mismatch-tolerant probe typically consists of a linear oligonucleotide that is perfectly complementary to the interrogated SNP allele. More specifically, the probe is typically designed to bind exclusively to the totality of perfectly matched SNP allele targets at a high temperature and to the totality of the two SNP allele variants of the same target at a sufficiently low temperature. The ratio of fluorescence signals at these two temperatures corrected for background probe signals collected at a third temperature where the probe does not bind to either allele target reflects the percentage of the interrogated SNP allele in the sample and represents molecular signatures unique to each genotype. Thus, following normalization for background signal differences among replicates and notwithstanding the quenching effect of temperature on fluorescence signals, heterozygous samples where the interrogated allele corresponds to 50% of the total amplification products generate half the fluorescence signal at high temperature and 100% of the fluorescence signal at the lower temperature. In contrast, homozygous samples consisting of 100% of the interrogated allele generate the same fluorescence signal at both high and low temperatures, see FIG. 2. Samples homozygous for the SNP allele that is not interrogated at the high temperature generate no fluorescent signal at high temperature and 100% of the fluorescent signal at the lower temperature, see FIG. 2.

LATE-PCR endpoint genotyping assays are robust because the fluorescence signal ratios associated with each genotype are an intrinsic thermodynamic property of the hybridization probe/target pair and are therefore independent of the amount of starting material in the amplification reaction or the amplification cycle chosen for end-point analysis during the linear phase of LATE-PCR. LATE-PCR endpoint genotyping assays also exhibit a greater multiplex capacity because, unlike traditional homogeneous genotyping methods that use fluorescent probes of different color for each allele, LATE-PCR endpoint assays can use a single fluorescent probe of any given color per SNP site.

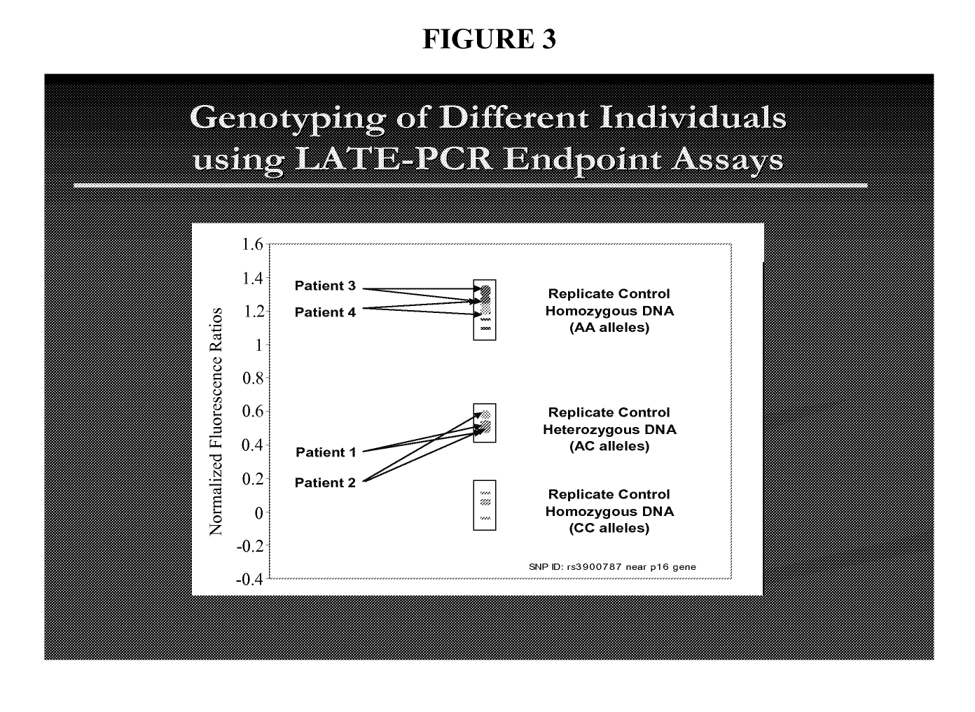

In practice, the fluorescent ratios associated with each genotype are first measured using replicate control DNA of known genotypes for the interrogated SNP allele. To account for variations, e.g., slight variations, in replicate fluorescent ratios due to sample-to-sample differences, the replicate fluorescent ratios from controls DNA samples are then used to define the 95% confidence interval for the range of fluorescent ratios associated with each genotype. Finally, genotype assignment for an unknown DNA sample is simply performed by measuring the fluorescent ratio of that sample and then determining into which 95% confidence interval of any given genotype the unknown fluorescent ratio falls into (see FIG. 3).

One issue for routine implementation of the LATE-PCR endpoint genotyping method is the variation in the 95% confidence intervals for the fluorescent ratios associated with each genotype in between experiments. Inter-assay variability of the 95% confidence intervals can sometimes make it difficult or impossible to define the 95% confidence intervals associated with each genotype in one experiment in advance, and then use those confidence intervals values for genotype assignment of fluorescent ratios from unknown samples in subsequent experiments. As a result, LATE-PCR endpoint genotyping may involve that the 95% confidence intervals for each genotype be established separately for every experiment. Since one typically uses 24 replicate control DNA for each genotype to define each 95% confidence intervals, one is forced to do 72 control DNA reactions (24 reactions for each of the possible three SNP site genotypes) in order to genotype a single unknown DNA sample.

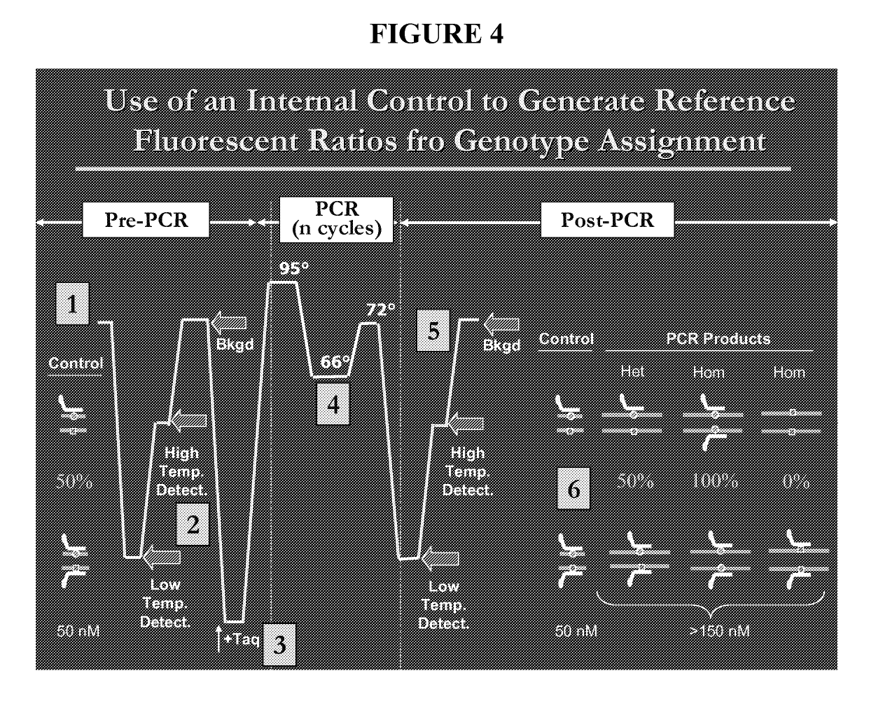

The systems and methods described herein may be used to avoid these issues. For example, the implementation of an internal control that is added to each unknown sample in order to generate a reference fluorescent ratio for genotype assignment allows for accurate target quantitation and/or instrument calibration without the need for replicate control DNA. In particular, this approach can circumvent multiple external DNA controls of known genotypes for the tested SNP allele and increases intra-assay as well as inter-assay reliability of LATE-PCR endpoint genotyping. The implementation of the methods in a LATE-PCR endpoint genotyping reaction is illustrated in FIG. 4.

An internal control template comprising an equimolar mixture of synthetic oligonucleotides corresponding to the matched and the mismatched probe target is added at low concentrations (e.g., 50 nM) to each PCR sample to simulate heterozygous control DNA (Step 1, FIG. 4). The PCR samples contain all the reagents except for DNA polymerase. In some embodiments, the added synthetic oligonucleotides are blocked at their 3′ end and are not complementary to the 3′ end of the amplification primers to prevent them from participating in the amplification reaction. Following binding of the mismatched—tolerant probe to the synthetic internal control oligonucleotides, the sample is heated up and fluorescent signals are collected at three different temperatures (Step 2, FIG. 4). These fluorescent signals are then used to determine the pre-PCR internal control fluorescence ratio corresponding to the heterozygous genotype for each particular PCR sample. Following addition of DNA polymerase (Step 3—FIG. 4) the sample is then subjected to PCR amplification (Step 3—FIG. 4). After PCR, the probe is annealed to the newly generated PCR products and the existing internal controls, heated up, and fluorescent signals are collected at three different temperatures to determine the post-PCR fluorescence ratio corresponding to the mixture of PCR products and the internal control (Step 5, FIG. 4). The pre-PCR fluorescent ratios are then subtracted from the post-PCR fluorescent ratios to obtain the fluorescent ratios derived from exclusively from the PCR products (Step 6, FIG. 4). Finally the adjusted post-PCR fluorescent ratio is compared to the pre-PCR internal control fluorescent ratio for genotype assignment. The calculation may be implemented manually or may be implemented automatically by a computing device. For example, a computing device may be configured to execute a program of instructions that cause the computing device to calculate the adjusted post-PCR fluorescent ratio. The instructions maybe embodied in a variety of computer-readable media, e.g. tangible media, non-transitory media and so on. In some embodiments, software is provided that instructs one or more computer processor to receive the quantitative information, and to calculate the ratios and/or provide a quantitative answer. In some embodiments, the processor is provided as part of the amplification instrumentation (e.g., thermocycler). However, the processor may be provided in any desired form (e.g., handheld computing device, remote computer, collaborative computing, over-the-cloud computing, etc.).

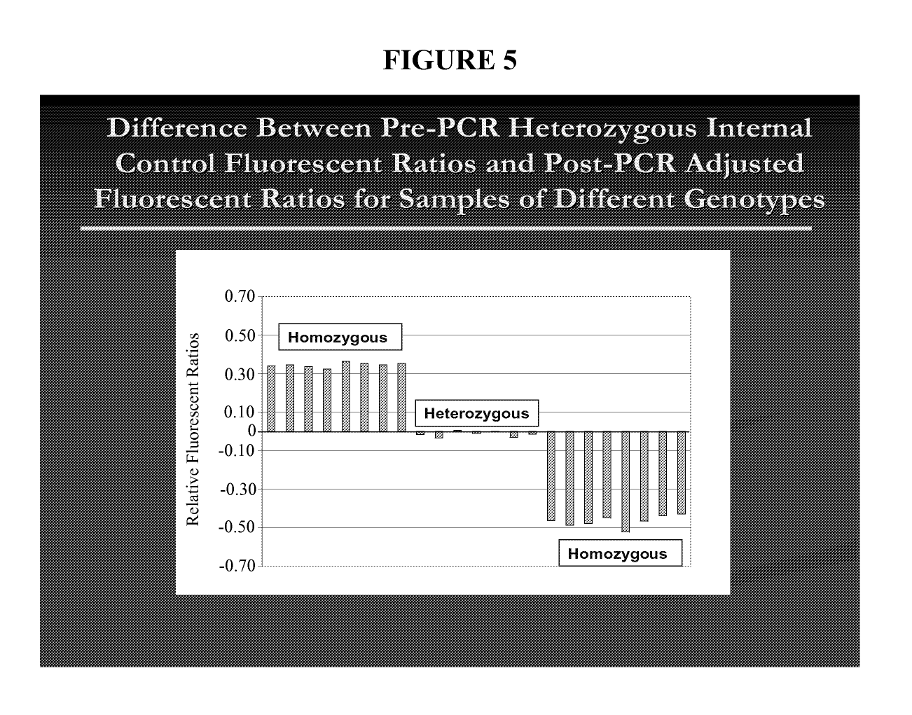

A sample heterozygous for the interrogated SNP allele will exhibit adjusted post-PCR fluorescent ratios that are approximately equal to the pre-PCR internal control fluorescent ratios. A sample homozygous for the interrogated SNP allele will exhibit adjusted post-PCR fluorescent ratios that are larger than the pre-PCR fluorescent ratios from the heterozygous internal control. A DNA sample homozygous for the SNP allele that is mismatched to the probe will exhibit adjusted post-PCR fluorescent ratios that are smaller than the pre-PCR fluorescent ratios from the heterozygous internal control, see FIG. 5

The above strategy may be use for a number of reasons. First, LATE-PCR fluorescent ratios are independent of the amount of target DNA in the reaction. As a result, fluorescent ratios obtained from 50 nM heterozygous internal control targets are the same as the fluorescent ratio obtained from the much more abundant PCR products at the end of the reaction (>150 nM). Second, internal control oligonucleotides can be constructed such that the fluorescent ratio for these templates matches the fluorescent ratio from amplified heterozygous DNA samples over a range of temperatures. This is despite differences in size and potential secondary structure between the internal control templates and the LATE-PCR amplification products. Third, the fluorescent ratios from internal controls oligonucleotides before LATE-PCR are not significantly altered following PCR amplification. This is despite the presence of DNA polymerase that binds the double-stranded probe-target hybrids and pH changes in the course of PCR amplification resulting the release of pyrophosphate following nucleotide incorporation into the amplifying DNA chains and PCR buffer breakdown following multiple cycles of heating and cooling in the course of LATE-PCR amplification.

The systems and methods provide a number of significant advantages. This strategy eliminates the use of multiple external DNA controls of known genotype for the interrogated SNP allele to define the 95% confidence intervals for the fluorescent ratios unique to each genotype. Each LATE-PCR sample has a built-in internal control that generates a reference heterozygous fluorescence ratio against which the fluorescent ratio from the PCR products is compared for genotype assignment. Additionally, since each sample is normalized against itself, this strategy corrects for difference normally found between replicate samples (e.g., differences associate with different well position in the PCR thermal cycle, use of different tubes, subtle differences in reaction conditions among replicate samples, etc). As a result, this strategy is contemplated to improve intra-assay fluorescent ratio reproducibility. By providing a built-in reference fluorescence ratio this approach is contemplated to also solve issues associated with inter-assay fluorescent ratio variability. Greater reproducibility of fluorescent ratio is contemplated to result in improved resolution of biological phenomena that result in quantitative alteration in fluorescent ratios (such as SNP allele imbalances resulting from loss of heterozygosity events). These benefits, while applicable to LATE-PCR, find general use across a wide variety of amplification reactions.

### Design Specifications of Illustrative Embodiments

Internal Control Oligonucleotides:

In some embodiments, the internal control comprise or consists of an equimolar mixture of synthetic oligonucleotides containing the matched and mismatched SNP allele targets of the mismatch-tolerant probe and is designed to simulate a heterozygous control DNA. Ideally, the internal control oligonucleotides generates the same fluorescent ratios as amplification products from genomic DNA encompassing the interrogated SNP site. The following criteria govern the design of the internal control oligonucleotides in some embodiments.
1. The internal control oligonucleotides should include the target site of the mismatched tolerant probe and may include any number of nucleotides flanking the 5′ end and/or the 3′ end probe target sequence in genomic DNA.
   a. Differences in the 5′ end or 3′ end target overhangs from the probe-target hybrid as well as differences in secondary structure between the amplicon and the internal control probe targets can cause fluorescent ratios differences between these two types of templates. The goal is to generate an internal control template that generates the same fluorescent ratio as the amplicon containing the interrogated SNP site. In most (but likely in not all cases) internal controls consisting of six nucleotides flanking sequence on either side of the probe target sequence work reasonably well.
2. The internal control oligonucleotides should not have any complementary to the 3′ end of the amplification primers that would result in extension of the primers on the probe sequence.
3. The internal control oligonucleotides should be blocked at the 3′ end to prevent them from acting as primers. Be aware that the presence of a 3′ end blocker such as a linker can affect the fluorescent ratios generated from these oligonucleotides at certain temperatures.

### Pre-PCR Steps

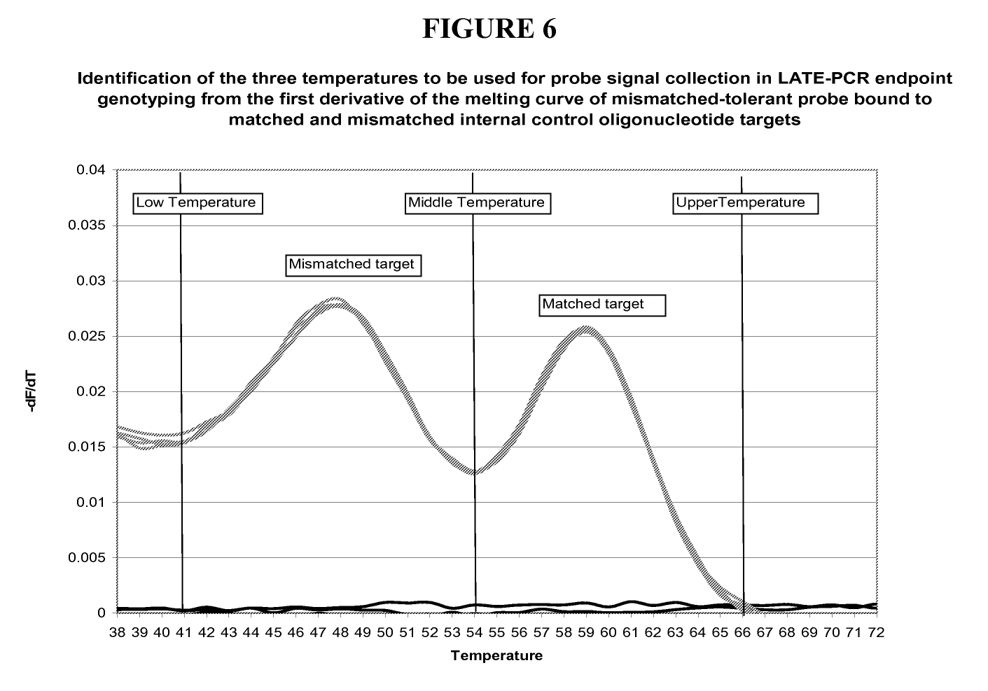

1. The internal controls may be added to each PCR sample at the lowest concentration that reliably generates fluorescent ratios. The goal is to prevent the internal control fluorescent signals from overwhelming the fluorescent signals from the PCR products at the end of the reaction. Experiments showed that 50 nM of each internal control oligonucleotides (the same concentration as the typical limiting primer concentration in LATE-PCR) works well.
2. In addition to the internal control, the PCR samples contain 1X PCR buffer, MgCl₂, dNTP, primers, probe (@500 nM), genomic DNA but no DNA polymerase during the pre-PCR steps.
   a. DNA polymerase is omitted from this step to prevent primer dimer formation during collection of fluorescent signals from the probe-internal control hybrids at three different temperatures.
3. As shown in FIG. 4, the sample is first heated to at least 10° C.-15° C. above the Tₘ of the probe bound to the matched target (i.e., a temperature where the internal control—probe hybrids are melted but genomic DNA is not denatured yet). The sample is then cooled gradually (0.1° C./sec) to a temperature at least 10° C.-15° C. below the Tₘ of the probe bound to the mismatched SNP allele target to allow complete probe target formation, although different cooling rates and ranges may be used.
4. The probe-internal control hybrids are then heated up at a fast rate (e.g., 2°-3° C./sec) at 1° C. intervals 30 seconds long up to at least 10° C.-15° C. above the Tₘ of the probe bound to the matched target and fluorescent signals are collected at three temperatures. Other rates and ranges of heating may be used as desired. The lowest temperature is the highest temperature at which the probe is bound to the totality of the matched and mismatched internal control targets. The middle temperature is the temperature where the probe is only detectably bound to the internal control matched targets. The upper temperature corresponds to the lowest temperature where the probe is not detectably bound to internal control targets. The actual temperatures to be used are identified from the 1^{st} derivative of the melting curve of probe-internal control target hybrids, as shown in FIG. 6.
5. Determination of fluorescent ratio also involves collection of fluorescent signals from samples with probe alone (i.e., no internal control template), at the same temperatures as above, to correct for the effect of temperature on fluorescent signal intensity. The fluorescent ratios from the fluorescent signal collected at these three temperatures are calculated from the formula

$$Ratio = \frac{( {[ IC ]}_{MT} * {[ Probe alone ]}_{HT} ) - ( {[ IC ]}_{HT} * {[ Probe alone ]}_{MT} )}{( {[ IC ]}_{LT} * {[ Probe alone ]}_{HT} ) - ( {[ IC ]}_{HT} * {[ Probe alone ]}_{LT} )}$$

Where IC_{LT}=fluorescent signal from the internal control at the low temperature.
   IC_{MT}=fluorescent signal from the internal control at the middle temperature
   IC_{HT}=fluorescent signal from the internal control at the high temperature
Probe Alone_{LT}=fluorescent signal from the internal control at the low temperature.
Probe Alone_{MT}=fluorescent signal from the internal control at the middle temperature
Probe Alone_{HT}=fluorescent signal from the internal control at the high temperature
6. The final pre-PCR step is to cool down the sample to room temperature (RT) and add polymerase to the sample

### Post-PCR Steps

1. Steps 4-5 performed during pre-PCR are done again post-PCR. The pre-PCR internal control fluorescent ratios are subtracted from the post-PCR fluorescent ratios on a per sample basis to obtain the fluorescent ratio values of the PCR products. The PCR product fluorescent ratio is then compared to the pre-PCR internal control ratios by subtraction for genotype assignment. If the PCR product fluorescent ratio is virtually identically to the pre-PCR fluorescent ratio the sample is then heterozygous for the interrogated SNP allele. If the PCR product fluorescent ratio is greater than the pre-PCR fluorescent ratio the sample is then homozygous for the interrogated SNP allele. If the PCR product fluorescent ratio is smaller than the pre-PCR fluorescent ratio the sample is then homozygous for the SNP allele that is not perfectly complementary to the mismatch tolerant probe.

In certain embodiments, the assays described herein employ primer pairs to amplify target nucleic acid sequences. The methods described herein are not limited by the type of amplification that is employed. In certain embodiments, PCR, asymmetric PCR, and/or LATE-PCR, is employed.

PCR is a repeated series of steps of denaturation, or strand melting, to create single-stranded templates; primer annealing; and primer extension by a thermally stable DNA polymerase such as Thermus aquaticus (Taq) DNA polymerase. A typical three-step PCR protocol may include denaturation, or strand melting, at 93-95 degrees C. for more than 5 sec, primer annealing at 55-65 degrees C. for 10-60 sec, and primer extension for 15-120 sec at a temperature at which the polymerase is highly active, for example, 72 degrees C. for Taq DNA polymerase. A typical two-step PCR protocol may differ by having the same temperature for primer annealing as for primer extension, for example, 60 degrees C. or 72 degrees C. For either three-step PCR or two-step PCR, an amplification involves cycling the reaction mixture through the foregoing series of steps numerous times, typically 25-40 times. During the course of the reaction the times and temperatures of individual steps in the reaction may remain unchanged from cycle to cycle, or they may be changed at one or more points in the course of the reaction to promote efficiency or enhance selectivity. In addition to the pair of primers and target nucleic acid a PCR reaction mixture typically contains each of the four deoxyribonucleotide 5′ triphosphates (dNTPs) at equimolar concentrations, a thermostable polymerase, a divalent cation, and a buffering agent. A reverse transcriptase is included for RNA targets, unless the polymerase possesses that activity. The volume of such reactions is typically 25-100 ul. Multiple target sequences can be amplified in the same reaction. In the case of cDNA amplification, PCR is preceded by a separate reaction for reverse transcription of RNA into cDNA, unless the polymerase used in the PCR possesses reverse transcriptase activity. The number of cycles for a particular PCR amplification depends on several factors including: a) the amount of the starting material, b) the efficiency of the reaction, and c) the method and sensitivity of detection or subsequent analysis of the product. Cycling conditions, reagent concentrations, primer design, and appropriate apparatuses for typical cyclic amplification reactions are well known in the art.

In one example, each strand of each amplicon molecule binds a primer at one end and serves as a template for a subsequent round of synthesis. The rate of generation of primer extension products, or amplicons, is thus generally exponential, theoretically doubling during each cycle. The amplicons include both plus (+) and minus (−) strands, which hybridize to one another to form double strands. To differentiate typical PCR from special variations described herein, typical PCR is referred to as “symmetric” PCR. Symmetric PCR thus results in an exponential increase of one or more double-stranded amplicon molecules, and both strands of each amplicon accumulate in equal amounts during each round of replication. The efficiency of exponential amplification via symmetric PCR eventually declines, and the rate of amplicon accumulation slows down and stops. Kinetic analysis of symmetric PCR reveals that reactions are composed of: a) an undetected amplification phase (initial cycles) during which both strands of the target sequence increase exponentially, but the amount of the product thus far accumulated is below the detectable level for the particular method of detection in use; b) a detected amplification phase (additional cycles) during which both strands of the target sequence continue to increase in parallel and the amount of the product is detectable; c) a plateau phase (terminal cycles) during which synthesis of both strands of the amplicon gradually stops and the amount of product no longer increases. Symmetric reactions slow down and stop because the increasing concentrations of complementary amplicon strands hybridize to each other (reanneal), and this out-competes the ability of the separate primers to hybridize to their respective target strands. Typically reactions are run long enough to guarantee accumulation of a detectable amount of product, without regard to the exact number of cycles needed to accomplish that purpose.

A technique that has found limited use for making single-stranded DNA directly in a PCR reaction is “asymmetric PCR.” Gyllensten and Erlich, “Generation of Single-Stranded DNA by the polymerase chain reaction and its application to direct sequencing of the HLA-DQA Locus,” Proc. Natl. Acad. Sci. (USA) 85: 7652 7656 (1988); Gyllensten, U. B. and Erlich, H. A. (1991) “Methods for generating single stranded DNA by the polymerase chain reaction” U.S. Pat. No. 5,066,584, Nov. 19, 1991; all of which are herein incorporated by reference. Asymmetric PCR differs from symmetric PCR in that one of the primers is added in limiting amount, typically 1/100th to ⅕th of the concentration of the other primer. Double-stranded amplicon accumulates during the early temperature cycles, as in symmetric PCR, but one primer is depleted, typically after 15-25 PCR cycles, depending on the number of starting templates. Linear amplification of one strand takes place during subsequent cycles utilizing the undepleted primer. Primers used in asymmetric PCR reactions reported in the literature, including the Gyllensten patent, are often the same primers known for use in symmetric PCR. Poddar (Poddar, S. (2000) “Symmetric vs. Asymmetric PCR and Molecular Beacon Probe in the Detection of a Target Gene of Adenovirus,” Mol. Cell Probes 14: 25 32 compared symmetric and asymmetric PCR for amplifying an adenovirus substrate by an end-point assay that included 40 thermal cycles. This paper reported that a primers ratio of 50:1 was optimal and that asymmetric PCR assays had better sensitivity that, however, dropped significantly for dilute substrate solutions that presumably contained lower numbers of target molecules. In some embodiments, asymmetric PCR is used with embodiments of the assays described herein.

In some embodiments, kits, compositions, and methods are based on Linear-After-The-Exponential (LATE) PCR (Pierce et al. Methods Mol Med. 2007; 132:65-85., herein incorporated by reference in its entirety), an advanced form of asymmetric PCR, that allows for rapid and sensitive detection at endpoint, together with PRIMESAFEII (Rice et al. Nat Protoc. 2007; 2(10):2429-38., herein incorporated by reference in its entirety), a PCR additive that maintains the fidelity of amplification over a broad range of target concentrations by suppressing mis-priming throughout the reaction. LATE-PCR assays reliably generate abundant single-stranded amplicons that can readily be detected in real-time and/or characterized at end-point using probes. In some embodiments, the assay functions as a duplex with an internal DNA control. The LATE-PCR assay described here can be used, for example, on standard laboratory equipment, and/or in the BIO-SEEQ Portable Veterinary Diagnostics Laboratory, a portable sample preparation and PCR instrument built by Smiths Detection. This device is specifically engineered for use in the field with a minimum of operator training. It includes an automated sample preparation unit that carries out sample preparation and LATE-PCR analysis on site in a matter of hours. Individual sample preparation units for the BIO-SEEQII, as well as the entire machine can be immersed in disinfectants (Virkon or Fam30) so as to ensure that contaminants (e.g. bacteria) is not transported away from the site of field testing.

The LATE-PCR assay is capable of detecting below 10 copies of a nucleic acid in clinical specimens. Since the assay is designed to be used, for example, in either laboratory settings or in a portable PCR machine (BIO-SEEQ Portable Veterinary Diagnostics Laboratory; Smiths Detection, Watford UK), the LATE-PCR provides a robust tool for the detection, identification, and analysis of NMD-1 variants, both in diagnostic institutes and in the field.

When using LATE-PCR, each reaction produces large amounts of specific, single-stranded DNA, which can then be probed with a sequence-specific probe. When tested against synthetic targets, the assay proved to be specific and effective even at low target numbers. Indeed, this assay generated robust specific signals down to approximately 1 molecule/reaction. The internal DNA control present in the assay is also specific and sensitive at low copy number.

LATE-PCR includes innovations in primer design, in temperature cycling profiles, and in hybridization probe design. Being a type of PCR process, LATE-PCR utilizes the basic steps of strand melting, primer annealing, and primer extension by a DNA polymerase caused or enabled to occur repeatedly by a series of temperature cycles. In the early cycles of a LATE-PCR amplification, when both primers are present, LATE-PCR amplification amplifies both strands of a target sequence exponentially, as occurs in conventional symmetric PCR. LATE-PCR then switches to synthesis of only one strand of the target sequence for additional cycles of amplification. In certain real-time LATE-PCR assays, the limiting primer is exhausted within a few cycles after the reaction reaches its C_{T} value, and in the certain assays one cycle after the reaction reaches its C_{T} value. As defined above, the C_{T} value is the thermal cycle at which signal becomes detectable above the empirically determined background level of the reaction. Whereas a symmetric PCR amplification typically reaches a plateau phase and stops generating new amplicons by the 50th thermal cycle, LATE-PCR amplifications do not plateau, because the do not continue to accumulate double-stranded products, and thus continue to generate single-stranded amplicons well beyond the 50th cycle, even through the 100th cycle. LATE-PCR amplifications and assays typically include at least 60 cycles, preferably at least 70 cycles when small (10,000 or less) numbers of target molecules are present at the start of amplification.

With certain exceptions, the ingredients of a reaction mixture for LATE-PCR amplification are generally the same as the ingredients of a reaction mixture for a corresponding symmetric PCR amplification. The mixture typically includes each of the four deoxyribonucleotide 5′ triphosphates (dNTPs) at equimolar concentrations, a thermostable polymerase, a divalent cation, and a buffering agent. As with symmetric PCR amplifications, it may include additional ingredients, for example reverse transcriptase for RNA targets. Non-natural dNTPs may be utilized. For instance, dUTP can be substituted for dTTP and used at 3 times the concentration of the other dNTPs due to the less efficient incorporation by Taq DNA polymerase.

In certain embodiments, the starting molar concentration of one primer, the “Limiting Primer,” is less than the starting molar concentration of the other primer, the “Excess Primer.” The ratio of the starting concentrations of the Excess Primer and the Limiting Primer is generally at least 5:1, preferably at least 10:1, and more preferably at least 20:1. The ratio of Excess Primer to Limiting Primer can be, for example, 5:1 . . . 10:1, 15:1 . . . 20:1 . . . 25:1 . . . 30:1 . . . 35:1 . . . 40:1 . . . 45:1 . . . 50:1 . . . 55:1 . . . 60:1 . . . 65:1 . . . 70:1 . . . 75:1 . . . 80:1 . . . 85:1 . . . 90:1 . . . 95:1 . . . or 100:1 . . . 1000:1 . . . or more. Primer length and sequence are adjusted or modified, preferably at the 5′ end of the molecule, such that the concentration-adjusted melting temperature of the Limiting Primer at the start of the reaction, T_{M[}0]^{L}, is greater than or equal (plus or minus 0.5 degrees C.) to the concentration-adjusted melting point of the Excess Primer at the start of the reaction, T_{M[}0]^{X}. Preferably the difference (T_{M[}0]^{L}−T_{M[}0]^{X}) is at least +3, and more preferably the difference is at least +5 degrees C.

Amplifications and assays according to embodiments of methods described herein can be performed with initial reaction mixtures having ranges of concentrations of target molecules and primers. LATE-PCR assays are particularly suited for amplifications that utilize small reaction-mixture volumes and relatively few molecules containing the target sequence, sometimes referred to as “low copy number.” While LATE-PCR can be used to assay samples containing large amounts of target, for example up to 10⁶ copies of target molecules, other ranges that can be employed are much smaller amounts, from to 1-50,000 copies, 1-10,000 copies and 1-1,000 copies. In certain embodiments, the concentration of the Limiting Primer is from a few nanomolar (nM) up to 200 nM. The Limiting Primer concentration is preferably as far toward the low end of the range as detection sensitivity permits.

In some embodiments compositions (e.g., kits, kit components, systems, instruments, reaction mixtures) comprising one or more or all of the components useful, necessary, or sufficient for carrying out any of the methods described herein are provided. In some embodiments, kits are provided containing one or more or all of the reagents.

The following Examples are presented in order to provide certain exemplary embodiments of the assays described herein and are not intended to limit the scope thereof.

### Example 1

### Use of Internal Control in Combination with LATE-PCR Endpoint Assay for Genotyping the rs8066665 SNP Site on Human Chromosome 17

The rs8066665 SNP site consists of a A-to-G polymorphism. DNA samples were obtained from the Coriell Cell Repository (Camdem, N.J.) from individuals of various genotypes for this SNP site (AA genotype—sample NA10855; AG genotype—sample NA10851; GG genotype—sample NA07348). The primers used to amplified genomic DNA encompassing this SNP site are

Limiting primer:
(SEQ ID NO: 1)
5′ GGAGGTCAGAGTACCCACTGCTCCTTC 3′
Excess primer:
(SEQ ID NO: 2)
5′ GCTCCTGAGCAATGAGAATGTC 3′

A mismatched tolerant probe was designed to be perfectly matched to the G allele (Tₘ=48° C.) and mismatched to the A allele (Tₘ=59° C.) (the Tₘ values correspond to 500 nM mismatch-tolerant probe and 50 nM matched or mismatched oligonucleotide targets).

probe:
(SEQ ID NO: 3)
5′ BHQ-1 CTTGCAGGTGGGATAGGA FAM 3′

The synthetic matched and mismatched targets for these probes are

matched target (30 nucleotides)

| | |
|---|---|
| 5′ GCATGCTCCTATCCCACCTGCAAGGGGTTG 3′ | (SEQ ID NO: 4) |

mismatched target (30 nucleotides)

| | |
|---|---|
| 5′ GCATGCTCCTATCCCACCTACAAGGGGTTG 3′ | (SEQ ID NO: 5) |

(Note: the highlighted nucleotide corresponds to the SNP allele on the target).

The Tₘ difference between the matched and mismatched targets allows the probe to bind to essentially 100% of the matched targets before it binds significantly to any mismatched target at a high temperature while also allowing probe binding to both matched and mismatched targets at a sufficiently low temperature.

Twenty LATE-PCR samples were set up comprising 1X PCR buffer, 3 mM MgCl₂, 0.25 mM dNTP, 25 mM PrimeSafe 060, 300 nM PrimeSafe 001, 50 nM limiting primer, 1 uM excess primer, 500 nM mismatched-tolerant probe. Sixteen of these samples contained 50 nM each of the matched and the mismatched oligonucleotide targets and the remaining four lacked these synthetic templates and serve as “probe alone” controls. Four of the sixteen samples containing probe plus IC control alone, but no genomic DNA. For the twelve remaining samples with internal control templates, sets of four samples received genomic DNA with different genotypes for the rs8066665 SNP site. All these samples were then heated to 72° C. to melt any pre-existing probe-target hybrids and then they were cooled to 30° C. at 0.1° C. per sec to allow formation of probe-target hybrid. The samples were heated up back to 72° C. at 1° C. intervals for 30 seconds each at a rate of 2° C.-3° C./sec. For the purpose of assay development fluorescent signals were collected at each of these intervals. After reaching 72° C. the samples were allowed to cool down to room temperature for addition of Taq DNA polymerase. Taq DNA polymerase was added to 1.25 units per LATE-PCR sample and the samples were subjected to LATE-PCR amplification for 70 cycles of 95° C. at 10 sec, 66° C. at 20 sec., and 72° C. at 20 sec. After PCR amplification the samples were cooled to 30° C. at 0.1° C. per sec to allow formation of probe-target hybrids. The samples were heated up back to 72° C. at 1° C. intervals for 30 seconds each at a rate of 2° C.-3° C./sec. For the purpose of assay development fluorescent signals were once against collected at each of these intervals.

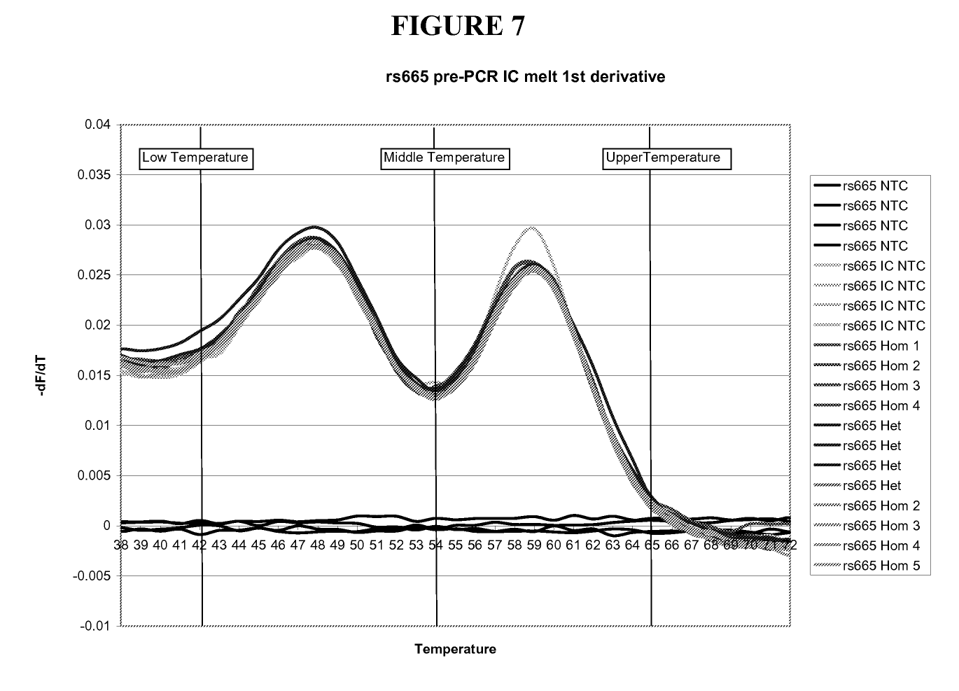

For pre-PCR fluorescent signal analysis, the first derivative of the melting curve was first calculated to identify the three temperatures needed for fluorescent signal normalization, as discussed above (see FIG. 7).

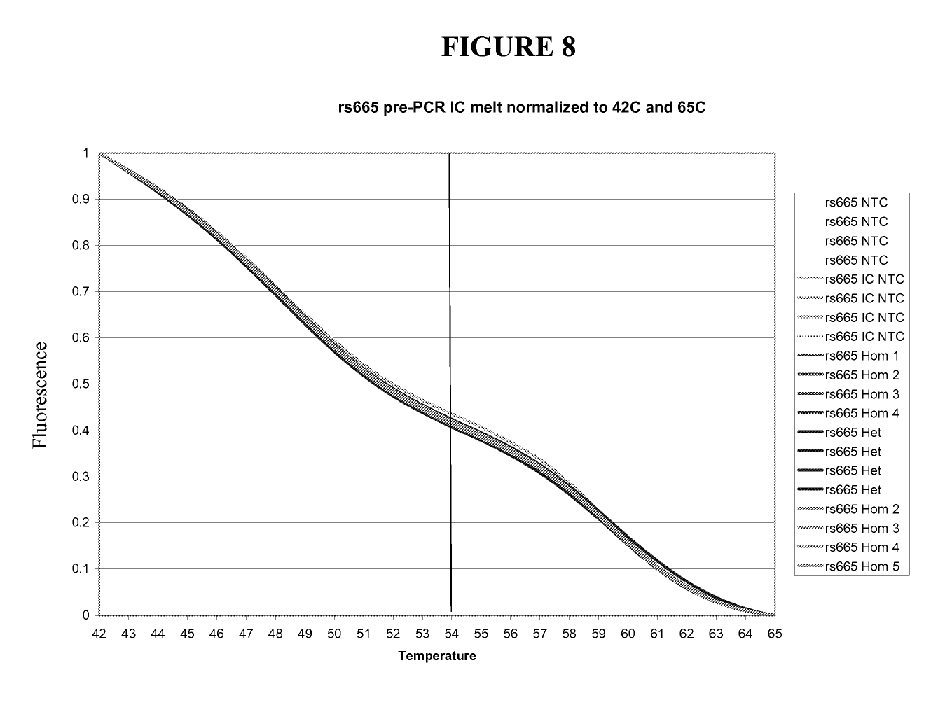

The raw fluorescent signals at the upper and the lower temperatures was then normalized to determine the fluorescent ratios for the internal control at all temperatures (see FIG. 8).

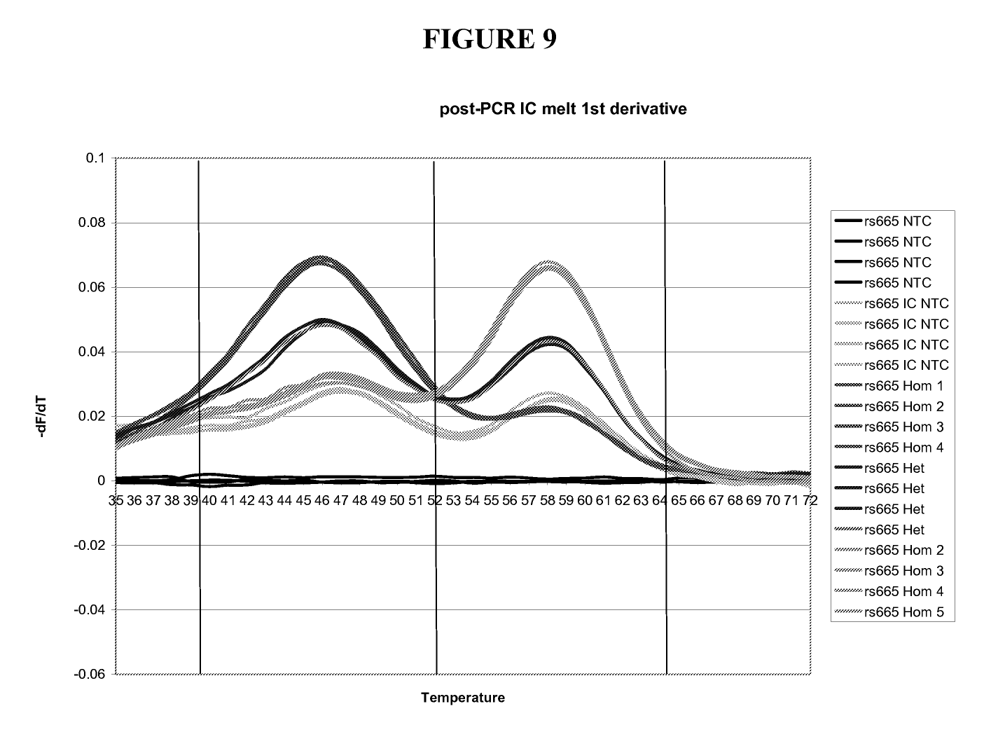

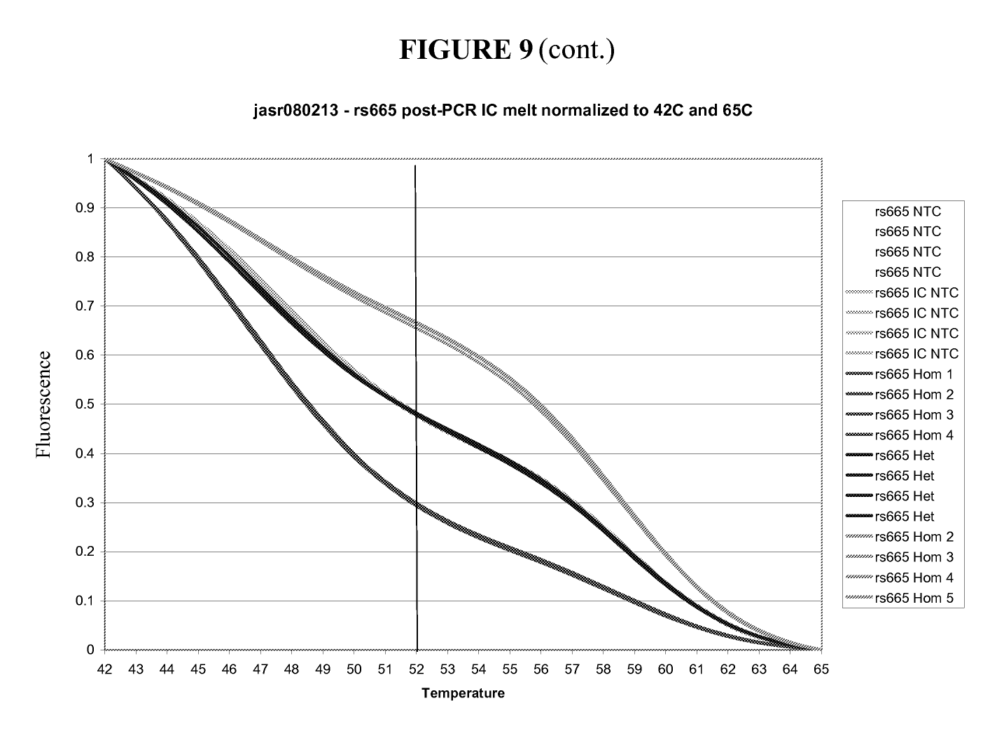

Following PCR the same 1^{st} derivative and fluorescent ratio analysis was performed (see FIG. 9).

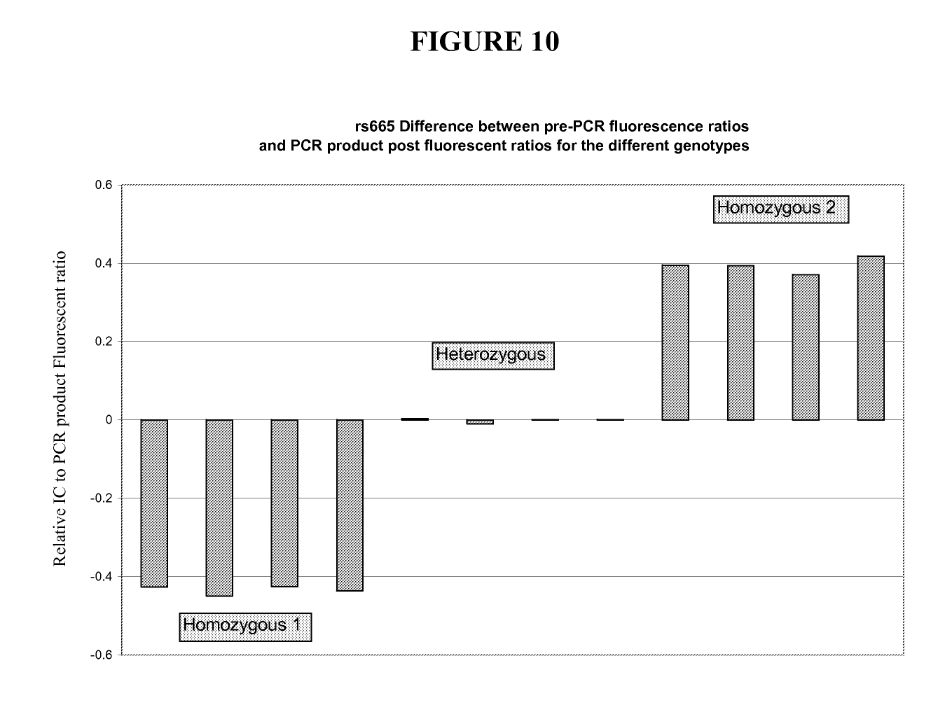

The pre-PCR fluorescent ratios was then subtracted from the post-PCR fluorescent ratios to obtain the fluorescent ratios derived exclusively from the PCR products and compared the resulting PCR products fluorescent ratio to the pre-PCR internal control fluorescence ratios by subtraction (see FIG. 10).

The results clearly show that the genotypes can be clearly identified on a per sample basis using the internal control as a reference.

All publications and patents mentioned in the present application are herein incorporated by reference. Various modification and variation of the described methods and compositions will be apparent to those skilled in the art without departing from the scope and spirit of the embodiments described herein. Although the methods, compositions, computer-readable media (including executable instructions), systems, and kits have been described in connection with specific exemplary embodiments, it should be understood that the claims should not be unduly limited to such specific embodiments. Indeed, various modifications of the described modes for carrying out the assays described herein that are obvious to those skilled in the relevant fields are intended to be within the scope of the following claims.

1.-19. (canceled)

20. A method for quantification of a nucleic acid amplification reaction, comprising:
(a) combining one or more target nucleic acids, amplification reagents, control nucleic acids, and one or more quantification probes in a reaction vessel;
(b) detecting the pre-amplification signal from the quantification probes at a high temperature and a low temperature;
(c) determining a pre-amplification ratio of the high temperature signal to the low temperature signal;
(d) detecting the post-amplification signal from the quantification probes at a high temperature and a low temperature;
(e) determining a post-amplification ratio of the high temperature signal to the low temperature signal;
(f) normalizing the post-amplification ratio to the pre-amplification ratio; and
(f) quantifying target amplification based on the normalized ratios.

21. The method of claim 20, wherein the nucleic acid amplification reaction comprises PCR.

22. The method of claim 21, wherein PCR comprises LATE-PCR.

23. The method of claim 20, wherein quantification probes comprise one or more detectable labels.

24. The method of claim 20, wherein quantification probes comprise one or more fluorescent labels.

25. The method of claim 20, wherein quantification probes comprise molecular beacons.

26. The method of claim 20, wherein obtaining temperature-dependent detection signatures comprises detecting one or more quantification probes at multiple temperatures.

27. The method of claim 20, wherein pre-amplification signal and post-amplification signal are detected at three temperatures.

28. The method of claim 20, wherein control nucleic acids are non-amplifiable in the amplification reaction.

29. The method of claim 20, wherein quantification probes are configured to hybridize to one or more sequences in the control nucleic acids.

30. The method of claim 20, wherein the quantification probes are configured to hybridize to sequences in two control nucleic acids at a low temperature.

31. The method of claim 20, wherein the quantification probes are configured to hybridize to sequences in one control nucleic acid at a high temperature that is higher than the low temperature.

32. The method of claim 20, wherein said quantification probes are configured not to hybridize to the control nucleic acids at a third temperature that is higher than the high temperature.

33. The method of claim 20, wherein the quantification probes are configured to hybridize with one or more sequences suspected to be present in the target nucleic acid or known to be present in the target nucleic acid. In some embodiments, target nucleic acids are amplifiable by the amplification reagents in an amplification reaction.

34. The method of claim 20, wherein normalizing comprises subtracting pre-amplification ratio from the post-amplification ratio to generate a normalized ratio.

35. The method of claim 20, wherein quantifying target amplification comprises comparing the normalized ratio to the pre-amplification ratio.

36. The method of claim 20, wherein pre-amplification ratios are obtained prior to the addition of the enzyme responsible for amplification to the reaction vessel.

37. A kit for performing the method of claim 20 comprising: amplification reagents, one or more quantification probes, and a set of control nucleic acids, wherein the control nucleic acids are non-amplifiable by the amplification reagents.

38. A system for analyzing the result of an amplification reaction comprising a processor configured for:
(a) accepting an input comprising:
(i) a first data set, wherein the first data set comprises pre-amplification ratios of a amplification reaction mixture; and
(ii) a second data set, wherein the second data set comprises post-amplification ratios of a amplification reaction mixture;
(b) calculating a normalized data set from the first data set and the second data set;
(c) generating results by comparing the second data set to the normalized data set; and
(d) reporting the results.
